(19) United States
(12) Patent Application Publication
(10) Pub. No.: US 2025/0046434 A1
(43) Pub. Date: Feb. 6, 2025

(54) MODELING OPERATIONAL BOUNDS OF LARGE LANGUAGE MODELS FOR THE MEDICAL DOMAIN

(71) Applicant: Siemens Healthineers AG, Forchheim (DE)

(72) Inventors: Bogdan Georgescu, Princeton, NJ (US); Oladimeji Farri, Upper Saddle River, NJ (US); Dorin Comaniciu, Princeton, NJ (US)

(21) Appl. No.: 18/363,087

(22) Filed: Aug. 1, 2023

(51) Int. Cl. G16H 40/20

(52) U.S. Cl. CPC G16H 40/20

(57) ABSTRACT

Systems and methods for determining an uncertainty measure associated with results of a medical task performed by an LLM (large language model) are provided. One or more prompts associated with a medical task are received. At least one of the one or more prompts are encoded into a set of features using a feature encoder network of an LLM. The medical task is performed based on the set of features using a decoder network of the LLM. An uncertainty measure associated with results of the medical task is determined based on the set of features using an uncertainty quantification module of the LLM. The results of the medical task and the uncertainty measure are output.

### TECHNICAL FIELD

The present invention relates generally to large language models, and in particular to modeling operational bounds of large language models for the medical domain.

### BACKGROUND

Generative AI (artificial intelligence) and LLMs (large language models) have received significant attention recently due to their ability to recognize, translate, predict, summarize, and generate text, programming code, images, or other content based on knowledge learned from very large amounts of training data. While generative abilities are a strength of conventional LLMs, it is also one of their weaknesses due to their tendency to fabricate or “hallucinate” information. Such hallucinations may occur when a query is received for information that is not present in the training data or the current prompt or when the query is ambiguous. Such hallucinations limit the applicability of LLMs to the medical domain and other critical decision-making applications.

### BRIEF SUMMARY OF THE INVENTION

In accordance with one or more embodiments, systems and methods for determining an uncertainty measure associated with results of a medical task performed by an LLM (large language model) are provided. One or more prompts associated with a medical task are received. At least one of the one or more prompts are encoded into a set of features using a feature encoder network of an LLM. The medical task is performed based on the set of features using a decoder network of the LLM. An uncertainty measure associated with results of the medical task is determined based on the set of features using an uncertainty quantification module of the LLM. The results of the medical task and the uncertainty measure are output.

In one embodiment, a distribution of a feature space of the LLM is modeled with a probability distribution function. The probability distribution function may comprise one of a Gaussian mixture model, a kernel density estimate of a Gaussian Process, or inducing points of a Gaussian Process. The probability distribution function may be computed over features of the LLM and image features extracted from the medical task images using a pre-trained model.

In one embodiment, an in-domain feature space of the LLM is modeled. An in-domain probability distribution function is generated over the in-domain feature space for the set of features and for a distribution of a feature space of the LLM. An out-of-domain probability distribution function is modelled as the complement of the in-domain probability distribution function. It is determined whether the set of features is out-of-domain of the LLM based on the in-domain probability distribution function and the out-of-domain probability distribution function.

In one embodiment, a notification is transmitted to a user to revise the one or more prompts based on the uncertainty measure.

In one embodiment, the results of the medical task are restricted based on the uncertainty measure.

In one embodiment, the uncertainty measure comprises at least one of a context confidence score or an answer confidence score.

In one embodiment, the LLM is constrained to a specific medical domain. The medical task may comprise at least one of summarizing one or more medical reports, determining a patient condition, and radiology reading assistance.

These and other advantages of the invention will be apparent to those of ordinary skill in the art by reference to the following detailed description and the accompanying drawings.

### BRIEF DESCRIPTION OF THE DRAWINGS

FIG. 1 shows a method for determining an uncertainty measure associated with results of a medical task generated by an LLM, in accordance with one or more embodiments;

FIG. 2 workflow for determining an uncertainty measure associated with results of a medical task generated by an LLM, in accordance with one or more embodiments;

FIG. 3 shows an exemplary artificial neural network that may be used to implement one or more embodiments;

FIG. 4 shows a convolutional neural network that may be used to implement one or more embodiments; and

FIG. 5 shows a high-level block diagram of a computer that may be used to implement one or more embodiments.

### DETAILED DESCRIPTION

The present invention generally relates to methods and systems for modeling operational bounds of LLMs for the medical domain. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

LLMs are deep learning models trained on a large language corpus of training data for recognizing, translating, predicting, summarizing, and generating text, programming code, images, and other forms of content. Conventional LLMs have a tendency to hallucinate information, limiting the applicability of conventional LLMs to the medical domain and other critical decision-making applications.

Embodiments described herein provide for LLMs implemented with an uncertainty quantification module to model the uncertainty of the LLMs in performing a particular medical task. The uncertainty may be modeled by fitting probability distribution models in the feature space (i.e., feature embedding space or latent feature space) of the LLMs. The uncertainty may be of various types, including: 1) epistemic or model uncertainty (uncertainty due to lack of knowledge about the best model to apply); 2) aleatoric or data uncertainty (uncertainty due to inherent randomness, such as, e.g., ambiguity in assigning class labels, measurement noise, homo/heteroscedastic noise, etc.); and 3) distributional uncertainty (uncertainty due to data distribution shifts between the training and the testing/validation datasets).

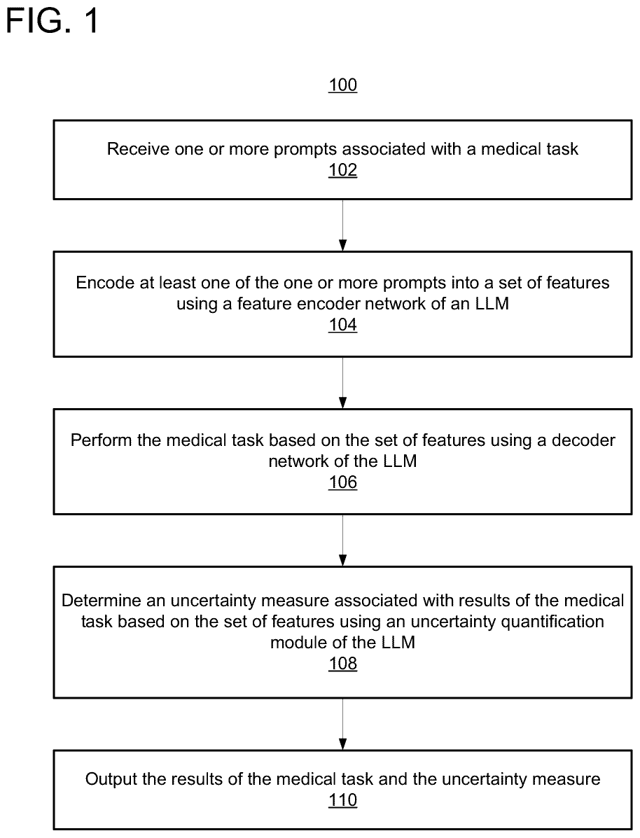

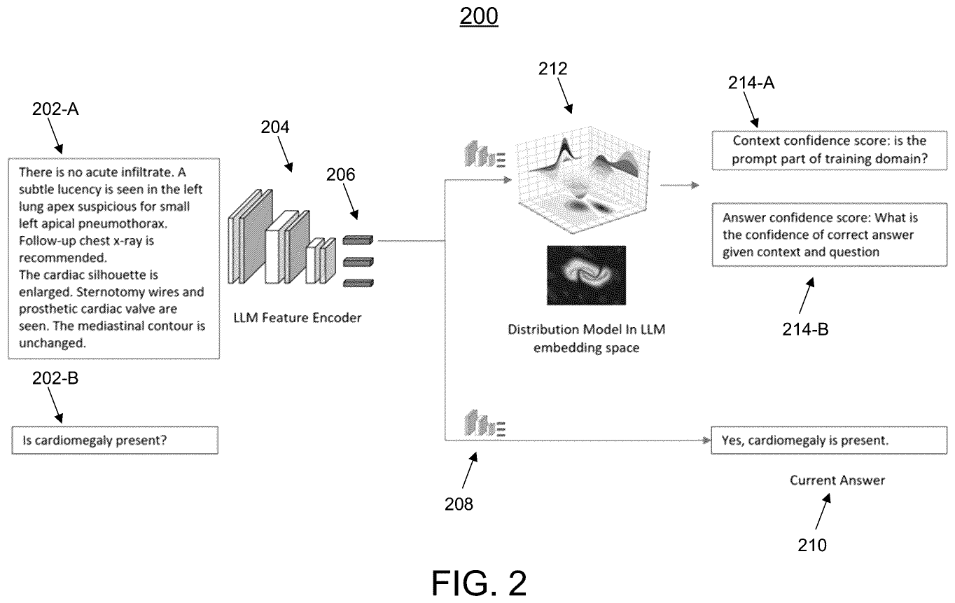

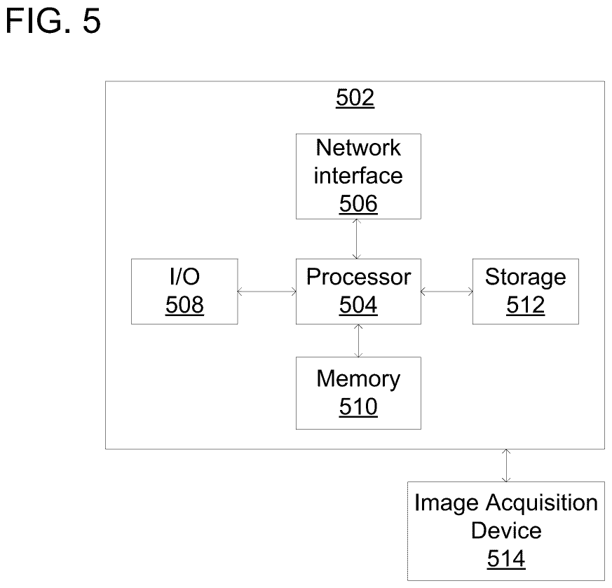

FIG. 1 shows a method 100 for determining an uncertainty measure associated with results of a medical task generated by an LLM, in accordance with one or more embodiments. The steps of method 100 may be performed by one or more suitable computing devices, such as, e.g., computer 502 of FIG. 5. FIG. 2 shows a workflow 200 for determining an uncertainty measure associated with results of a medical task generated by an LLM, in accordance with one or more embodiments. FIG. 1 and FIG. 2 will be described together.

At step 102 of FIG. 1, one or more prompts associated with a medical task are received. A prompt is a user provided input to an LLM from which the LLM is to perform the medical task. The prompt may include, for example, text-based instructions, questions, contextual information, and/or any other type of user input. The one or more prompts may be received from a computing device (e.g., computer 502 of FIG. 5) with which a user interacting.

In one example, as shown in workflow 200 of FIG. 2, the one or more prompts are prompts 202-A and 202-B (collectively referred to as prompts 202) received from a clinician (or any other user). Prompt 202-A comprises patient data to provide contextual information to the LLM. Prompt 202-B comprises a question or query to prompt the LLM to perform the medical task.

The medical task may be any suitable medical task which the LLM is prompted to perform. Exemplary medical tasks include summarizing medical reports (given a single or a plurality of (e.g., longitudinal) medical reports, determining patient condition given the current patient medical context, and radiology reading assistance. In the example shown in workflow 200 of FIG. 2, the medical task is determining whether cardiomegaly is present. It should be understood that the embodiments described herein is not limited to a medical task and that the embodiments described herein may be performed for any suitable task.

At step 104 of FIG. 1, at least one of the one or more prompts are encoded into a set of features using a feature encoder network of an LLM. The feature encoder network may be of any suitable architecture. The feature encoder network receives as input the at least one of the one or more prompts and generates as output the set of features. The set of features is a feature vector representing low level latent features or embeddings of the at least one of the one or more prompts. The feature space typically has a fixed size, which may be configurable as a parameter (i.e., number of tokens). In one example, as shown in workflow 200 of FIG. 2, the feature encoder network is LLM feature encoder 204 for encoding prompts 202-A to generate set of features 206.

The LLM may be any suitable pre-trained deep learning based LLM. For example, the LLM may be a transformer based LLM, such as, e.g., GPT (generative pre-training transformer)-based models, GPT-Neo, Bloom(z), BioGPT, Bert, etc. In one embodiment, as shown in FIGS. 1 and 2, the LLM is constrained to the medical domain for performing a medical task. The LLM may be constrained by, e.g., training, retraining, or refining the LLM for a specific medical domain on suitable training data. Embodiments described herein is not limited to the medical domain and the LLM may be constrained to any suitable target domain. The constraining of the LLM is not necessarily performed on training data from non-target (e.g., non-medical) domains, however such training data from non-medical domains may be used to ensure the LLM is discriminative in distinguishing the target domain.

At step 106 of FIG. 1, the medical task is performed based on the set of features using a decoder network of the LLM. The decoder may be of any suitable architecture. The decoder network receives as input the set of features and decodes the set of features to generate as output results of the medical task. In one example, as shown in workflow 200 of FIG. 2, the decoder network is decoder network 208, which receives as input the set of features 206 and generates as output results 210 of the medical task. Results 210 comprises an answer to the question of “is cardiomegaly present?” received via prompt 202-B.

At step 108 of FIG. 1, an uncertainty measure associated with results of the medical task is determined based on the set of features using an uncertainty quantification module of the LLM.

The uncertainty measure may comprise, e.g., a context confidence score and/or an answer confidence score. The context confidence score (e.g., for few-shot learning) represents the probability of the provided contextual information (received in the one or more prompts) belonging to the in-domain feature space of the LLM. This would indicate if the medical task was intended by the training constraints. The context confidence score may be based on the feature similarity between the question and contextual information provided in the one or more prompts and the results of the medical task. The answer confidence score (e.g., for zero-shot and few-shot learning) representing the probability of the answer being correct given the question and the contextual information (received in the one or more prompts).

In one example, as shown in workflow 200 of FIG. 2, the uncertainty quantification module is distribution model 212, which generates uncertainty measures 214-A and 214-B (collectively referred to as uncertainty measures 214) based on set of features 206. Uncertainty measure 214-A provides a context confidence score representing whether the prompt 202-B is part of the training domain. Uncertainty measure 214-B provides an answer confidence score representing the confidence in answer 210 being the correct answer given the contextual information in prompt 202-A and the question in prompt 202-B.

The uncertainty module models the distribution of the feature space of the LLM with a probability distribution function, such as, e.g., a GMM (Gaussian mixture model), KDE (kernel density estimate) of a Gaussian Process, inducing points of a Gaussian Process, etc. In one embodiment, an in-domain feature space of the LLM and an out-of-domain feature space of the LLM are modelled. The in-domain feature space corresponds to features of data on which the LLM is trained to perform a medical task and the out-of-domain feature space corresponds to features of data on which the LLM is not trained to perform a medical task. The feature space can optionally be made more descriptive when training, retraining, or refining the LLM by enforcing sensitivity and smoothness to reduce the potential influence of feature collapse. An in-domain probability distribution function is generated over the in-domain feature space for the set of features and for the distribution of the feature space of the LLM. An out-of-domain probability distribution function is modelled as the complement of the in-domain probability distribution function. It is then determined whether the set of features is out-of-domain of the LLM based on the in-domain probability distribution function and the out-of-domain probability distribution function.

In one embodiment, the uncertainty measure can be used to determine if the medical task was intended during training (i.e., out-of-domain detection) and restrict the answer given. In addition, low confidence (i.e., out-of-domain) uncertainty measures (e.g., as compared with a threshold) determined based on the distributional model of the LLM feature space may be leveraged as triggers to transmit a notification to the user for automated prompt revision towards increasing the similarity between the question and the contextual information (in the one or more prompts) within the feature space. In this manner, the LLM is self-correcting based on the uncertainty measure. In one embodiment, the LLM could output clarifying questions (if the LLM is fine-tuned for conversational tasks).

In one embodiment, the uncertainty measure may be used to generate a confidence measure for the “correctness” of the answer. The uncertainty measure allows the LLM to generate an answer only for high confidence cases, therefore increasing robustness and trust by allowing processing of information only in the target domain.

At step 110 of FIG. 1, the results of the medical task and/or the uncertainty measure are output. For example, the results of the medical task and/or the uncertainty measure can be output by displaying the results of the medical task and/or the uncertainty measure on a display device of a computer system, storing the results of the medical task and/or the uncertainty measure on a memory or storage of a computer system, or by transmitting the results of the medical task and/or the uncertainty measure to a remote computer system.

Advantageously, by modeling the feature space representing the operational bounds of the LLM, the LLM may be restricted to the intended target task (e.g., the medical task). This increases the ability of the LLM to distinguish between anomalies or non-intended tasks. Further, embodiments described herein reduce the impact of hallucinations generated by LLMs while providing a quantitative uncertainty measure. In addition, the uncertainty measure may be used to refine the LLMs on data where confidence/performance is lower than expected. The uncertainty measure may also be used in policy optimization in reinforcement learning setting towards auto-revising the prompt until a relatively better feature similarity between question and contextual information is achieved.

In one embodiment, multi-modal systems may be trained for image extracted features (e.g., using image-trained deep learning models) in addition to text extracted features with jointly trained probability distribution models.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, certain embodiments described herein are described with respect to methods and systems utilizing trained machine learning based models, as well as with respect to methods and systems for training machine learning based models. Features, advantages or alternative embodiments herein can be assigned to the other claimed objects and vice versa. In other words, claims for methods and systems for training a machine learning based model can be improved with features described or claimed in context of the methods and systems for utilizing a trained machine learning based model, and vice versa.

In particular, the trained machine learning based models applied in embodiments described herein can be adapted by the methods and systems for training the machine learning based models. Furthermore, the input data of the trained machine learning based model can comprise advantageous features and embodiments of the training input data, and vice versa. Furthermore, the output data of the trained machine learning based model can comprise advantageous features and embodiments of the output training data, and vice versa.

In general, a trained machine learning based model mimics cognitive functions that humans associate with other human minds. In particular, by training based on training data, the trained machine learning based model is able to adapt to new circumstances and to detect and extrapolate patterns.

In general, parameters of a machine learning based model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based model can be adapted iteratively by several steps of training.

In particular, a trained machine learning based model can comprise a neural network, a support vector machine, a decision tree, and/or a Bayesian network, and/or the trained machine learning based model can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

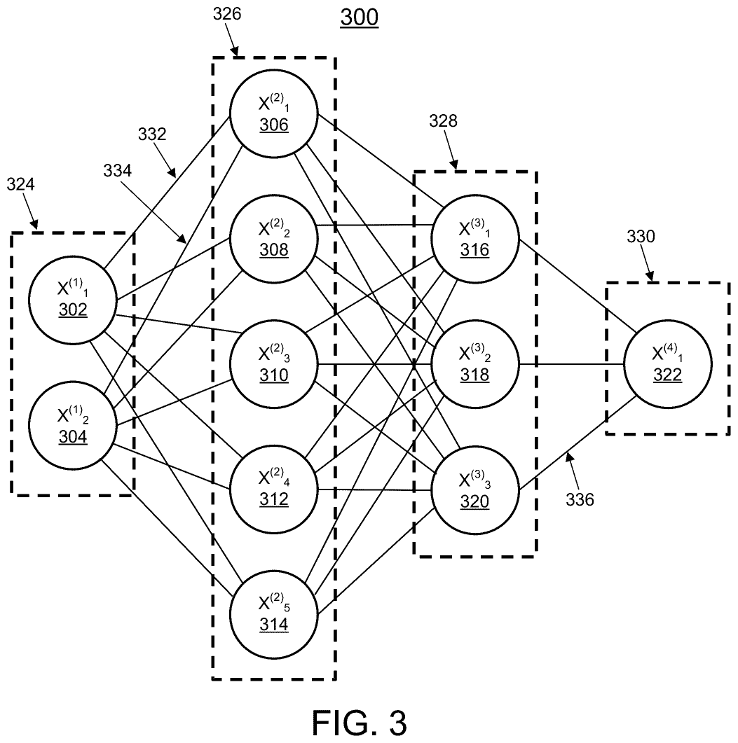

FIG. 3 shows an embodiment of an artificial neural network 300, in accordance with one or more embodiments. Alternative terms for “artificial neural network” are “neural network”, “artificial neural net” or “neural net”. Machine learning networks described herein, such as, e.g., the feature encoder network of the LLM utilized at step 104 and the decoder network of the LLM utilized at step 106 of FIG. 1 and LLM feature encoder 204 and decoder network 208 of FIG. 2, may be implemented using artificial neural network 300.

The artificial neural network 300 comprises nodes 302-322 and edges 332, 334, . . . , 336, wherein each edge 332, 334, . . . , 336 is a directed connection from a first node 302-322 to a second node 302-322. In general, the first node 302-322 and the second node 302-322 are different nodes 302-322, it is also possible that the first node 302-322 and the second node 302-322 are identical. For example, in FIG. 3, the edge 332 is a directed connection from the node 302 to the node 306, and the edge 334 is a directed connection from the node 304 to the node 306. An edge 332, 334, . . . , 336 from a first node 302-322 to a second node 302-322 is also denoted as “ingoing edge” for the second node 302-322 and as “outgoing edge” for the first node 302-322.

In this embodiment, the nodes 302-322 of the artificial neural network 300 can be arranged in layers 324-330, wherein the layers can comprise an intrinsic order introduced by the edges 332, 334, . . . , 336 between the nodes 302-322. In particular, edges 332, 334, . . . , 336 can exist only between neighboring layers of nodes. In the embodiment shown in FIG. 3, there is an input layer 324 comprising only nodes 302 and 304 without an incoming edge, an output layer 330 comprising only node 322 without outgoing edges, and hidden layers 326, 328 in-between the input layer 324 and the output layer 330. In general, the number of hidden layers 326, 328 can be chosen arbitrarily. The number of nodes 302 and 304 within the input layer 324 usually relates to the number of input values of the neural network 300, and the number of nodes 322 within the output layer 330 usually relates to the number of output values of the neural network 300.

In particular, a (real) number can be assigned as a value to every node 302-322 of the neural network 300. Here, x⁽ⁿ⁾ᵢ denotes the value of the i-th node 302-322 of the n-th layer 324-330. The values of the nodes 302-322 of the input layer 324 are equivalent to the input values of the neural network 300, the value of the node 322 of the output layer 330 is equivalent to the output value of the neural network 300. Furthermore, each edge 332, 334, . . . , 336 can comprise a weight being a real number, in particular, the weight is a real number within the interval [−1, 1] or within the interval [0, 1]. Here, w^{(m,n)}_{i,j} denotes the weight of the edge between the i-th node 302-322 of the m-th layer 324-330 and the j-th node 302-322 of the n-th layer 324-330. Furthermore, the abbreviation w⁽ⁿ⁾_{i,j} is defined for the weight w^{(n,n+1)}_{i,j}.

In particular, to calculate the output values of the neural network 300, the input values are propagated through the neural network. In particular, the values of the nodes 302-322 of the (n+1)-th layer 324-330 can be calculated based on the values of the nodes 302-322 of the n-th layer 324-330 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 324 are given by the input of the neural network 300, wherein values of the first hidden layer 326 can be calculated based on the values of the input layer 324 of the neural network, wherein values of the second hidden layer 328 can be calculated based in the values of the first hidden layer 326, etc.

In order to set the values w^{(m,n)}_{i,j} for the edges, the neural network 300 has to be trained using training data. In particular, training data comprises training input data and training output data (denoted as ti). For a training step, the neural network 300 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 300 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾ⱼ can be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 330, wherein f′ is the first derivative of the activation function, and y⁽ⁿ⁺¹⁾ⱼ is the comparison training value for the j-th node of the output layer 330.

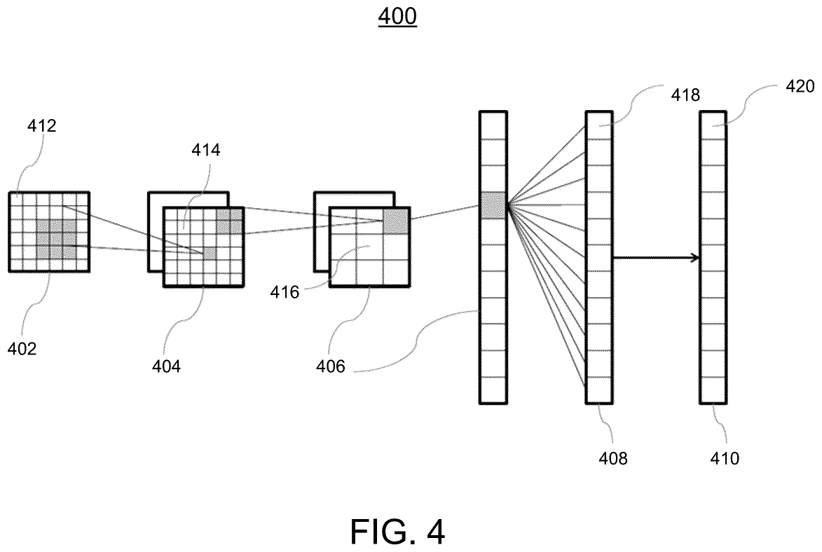

FIG. 4 shows a convolutional neural network 400, in accordance with one or more embodiments. Machine learning networks described herein, such as, e.g., the feature encoder network of the LLM utilized at step 104 and the decoder network of the LLM utilized at step 106 of FIG. 1 and LLM feature encoder 204 and decoder network 208 of FIG. 2, may be implemented using convolutional neural network 400.

In the embodiment shown in FIG. 4, the convolutional neural network comprises 400 an input layer 402, a convolutional layer 404, a pooling layer 406, a fully connected layer 408, and an output layer 410. Alternatively, the convolutional neural network 400 can comprise several convolutional layers 404, several pooling layers 406, and several fully connected layers 408, as well as other types of layers. The order of the layers can be chosen arbitrarily, usually fully connected layers 408 are used as the last layers before the output layer 410.

In particular, within a convolutional neural network 400, the nodes 412-420 of one layer 402-410 can be considered to be arranged as a d-dimensional matrix or as a d-dimensional image. In particular, in the two-dimensional case the value of the node 412-420 indexed with i and j in the n-th layer 402-410 can be denoted as x⁽ⁿ⁾_{[i,j]}. However, the arrangement of the nodes 412-420 of one layer 402-410 does not have an effect on the calculations executed within the convolutional neural network 400 as such, since these are given solely by the structure and the weights of the edges.

In particular, a convolutional layer 404 is characterized by the structure and the weights of the incoming edges forming a convolution operation based on a certain number of kernels. In particular, the structure and the weights of the incoming edges are chosen such that the values x⁽ⁿ⁾ₖ of the nodes 414 of the convolutional layer 404 are calculated as a convolution x⁽ⁿ⁾ₖ=Kₖ*x⁽ⁿ⁻¹⁾ based on the values x⁽ⁿ⁻¹⁾ of the nodes 412 of the preceding layer 402, where the convolution * is defined in the two-dimensional case as

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 412-418 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 412-420 in the respective layer 402-410. In particular, for a convolutional layer 404, the number of nodes 414 in the convolutional layer is equivalent to the number of nodes 412 in the preceding layer 402 multiplied with the number of kernels.

If the nodes 412 of the preceding layer 402 are arranged as a d-dimensional matrix, using a plurality of kernels can be interpreted as adding a further dimension (denoted as “depth” dimension), so that the nodes 414 of the convolutional layer 404 are arranged as a (d+1)-dimensional matrix. If the nodes 412 of the preceding layer 402 are already arranged as a (d+1)-dimensional matrix comprising a depth dimension, using a plurality of kernels can be interpreted as expanding along the depth dimension, so that the nodes 414 of the convolutional layer 404 are arranged also as a (d+1)-dimensional matrix, wherein the size of the (d+1)-dimensional matrix with respect to the depth dimension is by a factor of the number of kernels larger than in the preceding layer 402.

The advantage of using convolutional layers 404 is that spatially local correlation of the input data can exploited by enforcing a local connectivity pattern between nodes of adjacent layers, in particular by each node being connected to only a small region of the nodes of the preceding layer.

In embodiment shown in FIG. 4, the input layer 402 comprises 36 nodes 412, arranged as a two-dimensional 6×6 matrix. The convolutional layer 404 comprises 72 nodes 414, arranged as two two-dimensional 6×6 matrices, each of the two matrices being the result of a convolution of the values of the input layer with a kernel. Equivalently, the nodes 414 of the convolutional layer 404 can be interpreted as arranges as a three-dimensional 6×6×2 matrix, wherein the last dimension is the depth dimension.

A pooling layer 406 can be characterized by the structure and the weights of the incoming edges and the activation function of its nodes 416 forming a pooling operation based on a non-linear pooling function f. For example, in the two dimensional case the values x⁽ⁿ⁾ of the nodes 416 of the pooling layer 406 can be calculated based on the values x⁽ⁿ⁻¹⁾ of the nodes 414 of the preceding layer 404 as

$${x}^{( n )} [ i , j ] = f ( {x}^{( n - 1 )} [ {id}_{1} , {jd}_{2} ] , ... , {x}^{( n - 1 )} [ {id}_{1} + {d}_{1} - 1 , {jd}_{2} + {d}_{2} - 1 ] )$$

In other words, by using a pooling layer 406, the number of nodes 414, 416 can be reduced, by replacing a number d1-d2 of neighboring nodes 414 in the preceding layer 404 with a single node 416 being calculated as a function of the values of said number of neighboring nodes in the pooling layer. In particular, the pooling function f can be the max-function, the average or the L2-Norm. In particular, for a pooling layer 406 the weights of the incoming edges are fixed and are not modified by training.

The advantage of using a pooling layer 406 is that the number of nodes 414, 416 and the number of parameters is reduced. This leads to the amount of computation in the network being reduced and to a control of overfitting.

In the embodiment shown in FIG. 4, the pooling layer 406 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 408 can be characterized by the fact that a majority, in particular, all edges between nodes 416 of the previous layer 406 and the nodes 418 of the fully-connected layer 408 are present, and wherein the weight of each of the edges can be adjusted individually.

In this embodiment, the nodes 416 of the preceding layer 406 of the fully-connected layer 408 are displayed both as two-dimensional matrices, and additionally as non-related nodes (indicated as a line of nodes, wherein the number of nodes was reduced for a better presentability). In this embodiment, the number of nodes 418 in the fully connected layer 408 is equal to the number of nodes 416 in the preceding layer 406. Alternatively, the number of nodes 416, 418 can differ.

Furthermore, in this embodiment, the values of the nodes 420 of the output layer 410 are determined by applying the Softmax function onto the values of the nodes 418 of the preceding layer 408. By applying the Softmax function, the sum the values of all nodes 420 of the output layer 410 is 1, and all values of all nodes 420 of the output layer are real numbers between 0 and 1.

A convolutional neural network 400 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 400 can be trained based on the backpropagation algorithm. For preventing overfitting, methods of regularization can be used, e.g. dropout of nodes 412-420, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 502 that may be used to implement systems, apparatus, and methods described herein is depicted in FIG. 5. Computer 502 includes a processor 504 operatively coupled to a data storage device 512 and a memory 510. Processor 504 controls the overall operation of computer 502 by executing computer program instructions that define such operations. The computer program instructions may be stored in data storage device 512, or other computer readable medium, and loaded into memory 510 when execution of the computer program instructions is desired. Thus, the method and workflow steps or functions of FIG. 1 or 2 can be defined by the computer program instructions stored in memory 510 and/or data storage device 512 and controlled by processor 504 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 504 executes the method and workflow steps or functions of FIG. 1 or 2. Computer 502 may also include one or more network interfaces 506 for communicating with other devices via a network. Computer 502 may also include one or more input/output devices 508 that enable user interaction with computer 502 (e.g., display, keyboard, mouse, speakers, buttons, etc.).

Processor 504 may include both general and special purpose microprocessors, and may be the sole processor or one of multiple processors of computer 502. Processor 504 may include one or more central processing units (CPUs), for example. Processor 504, data storage device 512, and/or memory 510 may include, be supplemented by, or incorporated in, one or more application-specific integrated circuits (ASICs) and/or one or more field programmable gate arrays (FPGAs).

Data storage device 512 and memory 510 each include a tangible non-transitory computer readable storage medium. Data storage device 512, and memory 510, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 508 may include peripherals, such as a printer, scanner, display screen, etc. For example, input/output devices 508 may include a display device such as a cathode ray tube (CRT) or liquid crystal display (LCD) monitor for displaying information to the user, a keyboard, and a pointing device such as a mouse or a trackball by which the user can provide input to computer 502.

An image acquisition device 514 can be connected to the computer 502 to input image data (e.g., medical images) to the computer 502. It is possible to implement the image acquisition device 514 and the computer 502 as one device. It is also possible that the image acquisition device 514 and the computer 502 communicate wirelessly through a network. In a possible embodiment, the computer 502 can be located remotely with respect to the image acquisition device 514.

Any or all of the systems and apparatus discussed herein may be implemented using one or more computers such as computer 502.

One skilled in the art will recognize that an implementation of an actual computer or computer system may have other structures and may contain other components as well, and that FIG. 5 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising:
receiving one or more prompts associated with a medical task;
encoding at least one of the one or more prompts into a set of features using a feature encoder network of an LLM (large language model);
performing the medical task based on the set of features using a decoder network of the LLM;
determining an uncertainty measure associated with results of the medical task based on the set of features using an uncertainty quantification module of the LLM; and
outputting the results of the medical task and the uncertainty measure.

2. The computer-implemented method of claim 1, wherein determining an uncertainty measure associated with results of the medical task based on the set of features using an uncertainty quantification module of the LLM comprises:
modeling a distribution of a feature space of the LLM with a probability distribution function.

3. The computer-implemented method of claim 2, wherein the probability distribution function comprises one of a Gaussian mixture model, a kernel density estimate of a Gaussian Process, or inducing points of a Gaussian Process.

4. The computer-implemented method of claim 2, wherein the probability distribution function is computed over features of the LLM and image features extracted from the medical task images using a pre-trained model.

5. The computer-implemented method of claim 1, wherein determining an uncertainty measure associated with results of the medical task based on the set of features using an uncertainty quantification module of the LLM comprises:
modeling an in-domain feature space of the LLM;
generating an in-domain probability distribution function over the in-domain feature space for the set of features and for a distribution of a feature space of the LLM;
modeling an out-of-domain probability distribution function as the complement of the in-domain probability distribution function; and
determining whether the set of features is out-of-domain of the LLM based on the in-domain probability distribution function and the out-of-domain probability distribution function.

6. The computer-implemented method of claim 1, further comprising:
transmitting a notification to a user to revise the one or more prompts based on the uncertainty measure.

7. The computer-implemented method of claim 1, further comprising:
restricting the results of the medical task based on the uncertainty measure.

8. The computer-implemented method of claim 1, wherein the uncertainty measure comprises at least one of a context confidence score or an answer confidence score.

9. The computer-implemented method of claim 1, wherein the LLM is constrained to a specific medical domain.

10. The computer-implemented method of claim 1, wherein the medical task comprises at least one of summarizing one or more medical reports, determining a patient condition, and radiology reading assistance.

11. An apparatus comprising:
means for receiving one or more prompts associated with a medical task;
means for encoding at least one of the one or more prompts into a set of features using a feature encoder network of an LLM (large language model);
means for performing the medical task based on the set of features using a decoder network of the LLM;
means for determining an uncertainty measure associated with results of the medical task based on the set of features using an uncertainty quantification module of the LLM; and
means for outputting the results of the medical task and the uncertainty measure.

12. The apparatus of claim 11, wherein the means for determining an uncertainty measure associated with results of the medical task based on the set of features using an uncertainty quantification module of the LLM comprises:
means for modeling a distribution of a feature space of the LLM with a probability distribution function.

13. The apparatus of claim 12, wherein the probability distribution function comprises one of a Gaussian mixture model, a kernel density estimate of a Gaussian Process, or inducing points of a Gaussian Process.

14. The apparatus of claim 12, wherein the probability distribution function is computed over features of the LLM and image features extracted from the medical task images using a pre-trained model.

15. The apparatus of claim 11, wherein the means for determining an uncertainty measure associated with results of the medical task based on the set of features using an uncertainty quantification module of the LLM comprises:
means for modeling an in-domain feature space of the LLM;
means for generating an in-domain probability distribution function over the in-domain feature space for the set of features and for a distribution of a feature space of the LLM;
means for modeling an out-of-domain probability distribution function as the complement of the in-domain probability distribution function; and
means for determining whether the set of features is out-of-domain of the LLM based on the in-domain probability distribution function and the out-of-domain probability distribution function.

16. A non-transitory computer readable medium storing computer program instructions, the computer program instructions when executed by a processor cause the processor to perform operations comprising:
receiving one or more prompts associated with a medical task;
encoding at least one of the one or more prompts into a set of features using a feature encoder network of an LLM (large language model);
performing the medical task based on the set of features using a decoder network of the LLM;
determining an uncertainty measure associated with results of the medical task based on the set of features using an uncertainty quantification module of the LLM; and
outputting the results of the medical task and the uncertainty measure.

17. The non-transitory computer readable medium of claim 16, the operations further comprising:
transmitting a notification to a user to revise the one or more prompts based on the uncertainty measure.

18. The non-transitory computer readable medium of claim 16, the operations further comprising:
restricting the results of the medical task based on the uncertainty measure.

19. The non-transitory computer readable medium of claim 16, wherein the uncertainty measure comprises at least one of a context confidence score or an answer confidence score.

20. The non-transitory computer readable medium of claim 16, wherein the LLM is constrained to a specific medical domain.
